(19) United States
(12) Patent Application Publication
(10) Pub. No.: US 2017/0263879 A1
(43) Pub. Date: Sep. 14, 2017

(54) ORGANIC LIGHT EMITTING DIODE USING P-TYPE OXIDE SEMICONDUCTOR CONTAINING GALLIUM, AND PREPARATION METHOD THEREFOR

(71) Applicant: UNIVERSITY-INDUSTRY COOPERATION GROUP OF KYUNG HEE UNIVERSITY, Yongin-si, Gyeonggi-do (KR)

(72) Inventors: Jin JANG, Seoul (KR); Jeong Gi KIM, Seoul (KR); Christophe Vincent AVIS, Seoul (KR)

(21) Appl. No.: 15/510,416

(22) PCT Filed: Sep. 11, 2015
(86) PCT No.: PCT/KR2015/009586

§ 371 Date: Mar. 10, 2017

(30) Foreign Application Priority Data
Sep. 11, 2014 (KR) 10-2014-0120365

(51) Int. Cl. H01L 51/50; C09D 1/00; C09D 11/52; C09D 11/037; H01L 51/52; C09D 11/322; C09D 11/36; H01L 51/56; H01L 51/00; C09D 5/24; C09D 11/033

(52) U.S. Cl. CPC H01L 51/5088; C09D 5/24; C09D 1/00; C09D 11/52; C09D 11/037; C09D 11/033; C09D 11/322; C09D 11/36; H01L 51/56; H01L 51/001; H01L 51/0021; H01L 51/0003; H01L 51/5056; H01L 51/5012; H01L 51/5072; H01L 51/5206; H01L 51/5221; H01L 2251/303

(57) ABSTRACT

The present invention relates to an organic light emitting diode using a p-type oxide semiconductor containing gallium, and a preparation method therefor. According to the present invention, provided is an organic light emitting diode comprising an anode, a hole injection layer, a hole transport layer, a light emitting layer, an electron transport layer and a cathode, wherein the hole injection layer is a p-type oxide semiconductor containing Ga.

### CROSS-REFERENCE TO RELATED APPLICATIONS

This application is a National Phase Application of PCT International Application No. PCT/KR2015/009586, which was filed on Sep. 11, 2015, and which claims priority from Korean Patent Application No. 10-2014-0120365 filed with the Korean Intellectual Property Office on Sep. 11, 2014. The disclosures of the above patent applications are incorporated herein by reference in their entirety.

### BACKGROUND

1. Technical Field

The present invention relates to an organic light emitting diode (OLED) using a p-type oxide semiconductor containing gallium and to a method for preparing the same.

2. Description of the Related Art

There are continued development efforts aimed at manufacturing organic light emitting diodes of high efficiency.

In this regard, the movement of holes is a very important aspect. While the PEDOT:PSS (Poly(3,4-ethylenedioxythiophene) poly(styrenesulfonate)) layer is used as a typical hole injection layer, it entails limits in terms of the injection and movement of holes and the efficiency of the OLED.

Also, when PEDOT:PSS is used as a hole injection layer, an additional annealing time may be needed, resulting in increased process times.

Meanwhile, research is under way aimed at replacing the hole injection layer with an oxide semiconductor. This is because an oxide semiconductor not only offers greater mobility and transparency, making it possible to implement a transparent display more easily, but also is recognized as alternative technology that can overcome the limitations found in existing technology.

In addition, since it has an amorphous or polycrystalline structure at normal temperature, it does not require a separate heat treatment process for forming grains, and provides desirable properties when applied to an OLED.

However, oxide semiconductors are mainly reported as n-types due to oxygen vacancies and zinc interstitials, and p-type doping may be difficult to implement.

Since most hitherto known oxide semiconductors thus show n-type properties, and since a transparent oxide semiconductor having p-type properties would provide many advantages as a hole injection layer for an OLED, there is a need for more research on finding p-type transparent oxide semiconductor materials, for example by adjusting doping conditions or developing new substances, etc.

### SUMMARY OF THE INVENTION

An aspect of the invention is to provide an organic light emitting diode (OLED) using a p-type oxide semiconductor that contains gallium and a method of preparing the OLED.

Other objectives of the present invention can be derived by the skilled person from the embodiments described herein.

The present invention was conceived to resolve the problems found in the related art described above, and an embodiment of the invention provides an OLED that includes an anode, a hole injection layer, a hole transport layer, a light emitting layer, an electron transport layer, and a cathode, wherein the hole injection layer is a p-type oxide semiconductor containing Ga.

The p-type oxide semiconductor can include Ga contained in CuS and SnO.

The content of the Ga can be within a range of 10 to 70% (atomic percent) of the total composition.

The p-type oxide semiconductor can include one or more compound expressed by any one or more of Formula 1, Formula 2, and Formula 3 below:

CuS₁₋ₓGaₓ—SnO [Formula 1]

CuSGaₓSn₁₋ₓO [Formula 2]

CuSGaₓSnO [Formula 3]

where 0<x<1 in Formula 1, Formula 2, or Formula 3.

The hole injection layer can be heat treated at a predetermined temperature or can be UV treated.

The heat treatment temperature for the hole injection layer can be within a range of 150 to 250° C.

Another embodiment of the invention provides an OLED that includes an anode, a hole injection/transport layer, a light emitting layer, an electron transport layer, and a cathode, wherein the hole injection/transport layer is a p-type oxide semiconductor containing Ga.

Still another embodiment of the invention provides a preparation method for an OLED that includes: forming an anode on a substrate by way of a vacuum deposition process; forming a hole injection layer on the anode by way of a solution process; forming a hole transport layer on the hole injection layer by way of a vacuum deposition process; forming a light emitting layer on the hole transport layer by way of a vacuum deposition process; forming an electron transport layer on the light emitting layer by way of a vacuum deposition process; and forming a cathode on the electron transport layer, wherein the hole injection layer is formed by forming a membrane from a solution containing a p-type oxide semiconductor mixed into a solvent.

According to an embodiment of the invention, a p-type oxide semiconductor that contains gallium may be provided as a hole injection layer, making it possible to implement an OLED having high efficiency.

Also, according to an embodiment of the invention, a p-type oxide semiconductor prepared using a solution process can be utilized, whereby preparation is possible at lower temperatures and with lower costs.

Additional aspects and advantages of the present invention will be set forth in part in the description which follows, and in part will be obvious from the description, or may be learned by practice of the invention.

### BRIEF DESCRIPTION OF THE DRAWINGS

FIG. 1 is a cross-sectional diagram illustrating the structure of an OLED according to an embodiment of the invention.

FIG. 2A to FIG. 2E illustrate the surfaces of thin films using a p-type oxide semiconductor according to an embodiment of the invention and PEDOT:PSS.

FIG. 3 shows XRD (X-ray diffraction) results for cases using a p-type oxide semiconductor according to an embodiment of the invention and a case using PEDOT:PSS.

FIG. 4A to FIG. 4E show current-voltage-luminance properties of OLED's for cases using a p-type oxide semiconductor according to an embodiment of the invention and a case using PEDOT:PSS.

FIG. 5A to FIG. 5C show spectrum properties of OLED's for cases using a p-type oxide semiconductor according to an embodiment of the invention and a case using PEDOT:PSS.

FIG. 6 shows external quantum efficiency properties of OLED's for cases using a p-type oxide semiconductor according to an embodiment of the invention and a case using PEDOT:PSS.

FIG. 7 shows life expectancy properties of OLED's for cases using a p-type oxide semiconductor according to an embodiment of the invention and a case using PEDOT:PSS.

FIG. 8 shows XRD results when the concentration of Ga in a CuS—GaₓSn₁₋ₓO thin film is 0˜50%.

FIG. 9A to FIG. 9C show AFM images when the concentration of Ga in a CuS—GaₓSn₁₋ₓO thin film is 0%, 30%, and 50%.

FIG. 10A and FIG. 10B show (a) a TEM image and (b) atomic mapping images obtained by EDX (energy dispersive X-ray) spectroscopy for a CuS—SnO thin film.

FIG. 11A and FIG. 11B show (a) a TEM image and (b) atomic mapping images obtained by EDX (energy dispersive X-ray) spectroscopy for a CuS—Ga_{0.5}Sn_{0.5}O thin film.

FIG. 12 shows values of the UPS (ultraviolet photospectroscopy) and work function, the difference between the Fermi level and valence band, and the ionization potential of SnO₂ and CuS—GaₓSn₁₋ₓO.

FIG. 13A and FIG. 13B show Raman spectrum results of p-type precursor solutions.

### DETAILED DESCRIPTION OF THE INVENTION

First, the definitions of some of the terms used in the present specification are provided below.

A solution process includes any existing process for forming a membrane using a liquid solvent, such as spin coating, spray coating, deep coating, inkjet printing, roll-to-roll printing, screenprinting, etc.

A vacuum deposition process refers to a process in which deposition is performed while a negative pressure is applied, and includes any existing process such as CVD (chemical vapor deposition) and sputtering, a type of PVD (physical vapor deposition).

The present invention is described below in more detail with reference to the drawings. However, it is to be appreciated that the drawings are provided merely to facilitate the description of the invention and that they do not limit the scope of invention.

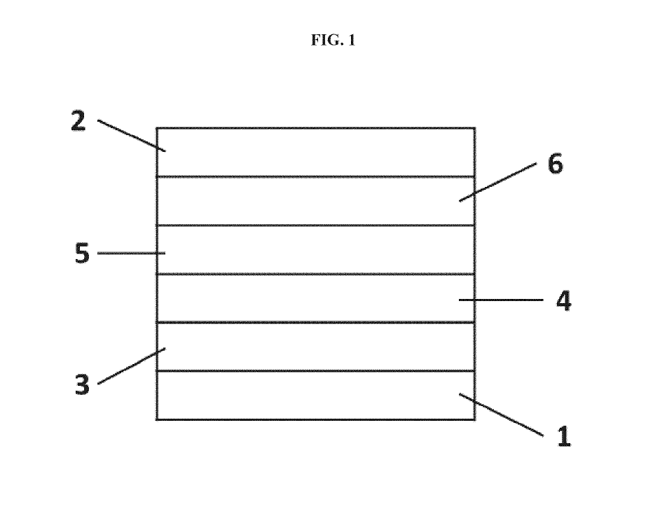

FIG. 1 is a cross-sectional diagram illustrating the structure of an OLED according to an embodiment of the invention.

As illustrated in FIG. 1, an organic light emitting diode (OLED) according to an embodiment of the invention can include an anode 1, a cathode 2, a hole injection layer 3, a hole transport layer 4, a light emitting layer 5, and an electron transport layer 6.

The anode 1 and cathode 2 can be formed using well-known methods such as by a vacuum deposition process (CVD) or by printing a metal paste ink in which metal flakes or particles are mixed with a binder. The method of forming the anode or cathode is not limited to a particular technique.

The cathode, formed on a substrate, is the electrode that provides electrons to the component and can be made from an ionized metallic substance, a metallic ink substance in a colloid state within a particular liquid, a transparent metal oxide, and the like.

The substrate can be a glass substrate, a plastic substrate that includes any plastic containing PET (polyethylene terephthalate), PEN (polyethylene naphthalate), PP (polypropylene), PI (polyamide), TAC (triacetyl cellulose), PES (polyethersulfone), etc., a flexible substrate that includes any one of aluminum foil and stainless steel foil, and the like.

The cathode 2 can be deposited in a high-vacuum state by a vacuum deposition process or can also be formed by applying a metallic substance used in the related art for forming cathodes in the form of a solution or a paste. There is no particular limit to the substance that may be used for forming the cathode, and substances used in the related art for forming cathodes can be used without limit. Examples of substances used in the related art for forming cathodes include metals that can be easily oxidized, such as aluminum (Al), calcium (Ca), barium (Ba), magnesium (Mg), lithium (Li), cesium (Cs), etc.

Also, some non-limiting examples of transparent metal oxides that can be used for forming the cathode include ITO (indium tin oxide), FTO (fluorine-doped tin oxide), ATO (antimony tin oxide), AZO (aluminum-doped zinc oxide), etc. In the case of a transparent metal oxide electrode, a process such as sol-gel, spray pyrolysis, sputtering, ALD (atomic layer deposition), e-beam evaporation, etc., can be used.

The electron transport layer 6 is the layer added to provide high efficiency for the component by moving the electrons generated at the cathode 2 to the light emitting layer 5 and may be formed between the cathode 2 and the light emitting layer 5.

The electron transport layer 6 can be formed by applying a vacuum deposition process using an organic substance in a high-vacuum state.

The light emitting layer 5 may include an organic substance and may generate light by way of a photoemission effect undergone by the organic substance.

The hole transport layer 4 is the layer that aids the moving of holes, which are injected at the hole injection layer 3, towards the light emitting layer 5 and may be formed between the light emitting layer 5 and the hole injection layer 3.

The hole transport layer 4 can be formed by applying a vacuum deposition process using an organic substance in a high-vacuum state.

The hole injection layer 3 is the layer that aids the moving of holes, which are injected at the anode 1, towards the hole transport layer 4 and may be formed between the hole transport layer 4 and the anode 1.

According to a preferred embodiment of the invention, the hole injection layer 3 may be formed using a p-type oxide semiconductor instead of the generally used PEDOT:PSS.

While FIG. 1 illustrates the hole injection layer 3 and the hole transport layer 4 as separated forms, and while this embodiment is described with reference to the hole injection layer 3 formed from a p-type oxide semiconductor, the invention is not limited thus, and the case of forming a hole injection/transport layer from a p-type oxide semiconductor with a single layer for the injection and movement of holes can be encompassed within the scope of the invention.

Preferably, the p-type oxide semiconductor can contain gallium (Ga), where the content of gallium in the p-type oxide semiconductor can be within a range of 10 to 70 atomic percent.

According to an embodiment of the invention, the p-type oxide semiconductor can be formed by way of a solution process, and here, the solvent can include acetonitrile mixed in a proportion of 5 to 50 volume percent into ethylene glycol.

According to another embodiment of the invention, at least one of DI water, alcohol, cyclohexane, toluene, and an organic solvent can be used besides acetonitrile.

A p-type oxide semiconductor according to a preferred embodiment of the invention can be formed by bonding between CuS and one or more substances selected from SnO, ITO, IZTO, IGZO, and IZO, and with an additional bonding by Ga.

CuS refers to copper monosulfide, SnO refers to tin (II) oxide, ITO refers to indium tin oxide, IZTO refers to indium zinc tin oxide, IGZO refers to indium gallium zinc oxide, and IZO refers to indium zinc oxide. These terms are obvious to those of ordinary skill in the relevant field of art (hereinafter referred to as the ‘skilled person’).

A p-type oxide semiconductor according to a preferred embodiment of the invention can be such that is expressed by one or more of Formula 1, Formula 2, and Formula 3 shown below.

CuS₁₋ₓGaₓ—SnO [Formula 1]

CuSGaₓSn₁₋ₓO [Formula 2]

CuSGaₓSnO [Formula 3]

In Formula 1, Formula 2, or Formula 3 above, 0<x<1.

A p-type oxide semiconductor according to an embodiment of the invention can be formed by sequentially performing the operations of:

preparing a precursor solution containing Cu, S, M, and Ga (where M is one or more compound selected from a group consisting of SnO, ITO, IZTO, IGZO, and IZO);

coating the precursor solution on a substrate; and

heat treating the coating layer.

The precursor solution may preferably contain [CuTu₃]Cl.

The precursor solution may preferably contain Thiourea.

The operation of coating onto the substrate can be based on a vacuum process, spin coating, slot printing, or inkjet printing, but in terms of the simplicity and cost of the process, it may be preferably to use a spin coating or an inkjet printing process.

The anode 1 is the electrode that provides holes to the component and can be formed by a solution process such as screenprinting, etc., a metal paste or a metal ink substance of a colloidal state in a particular liquid. Here, the metal paste can be any one of silver (Ag) paste, aluminum (Al) paste, gold (Au) paste, copper (Cu) paste, etc., or an alloy thereof. Also, the metal ink substance can be at least one of silver (Ag) ink, aluminum (Al) ink, gold (Au) ink, calcium (Ca) ink, magnesium (Mg) ink, lithium (Li) ink, and cesium (Cs) ink. The metal substances contained in the metal ink substance may be in an ionized state in a solution.

A detailed description has been provided above of the structure of an OLED based on an embodiment of the invention. According to other preferred embodiments of the invention, a hole injection layer formed from a p-type oxide semiconductor can be applied not only to an OLED that includes a light emitting layer but also to other organic electrical components.

A preparation method for an organic light emitting diode (OLED) according to an embodiment of the invention may include the operations of:

forming an anode on a substrate by way of a vacuum deposition process;

forming a hole injection layer on the anode by way of a solution process;

forming a hole transport layer on the hole injection layer by way of a vacuum deposition process;

forming a light emitting layer on the hole transport layer by way of a vacuum deposition process;

forming an electron transport layer on the light emitting layer by way of a vacuum deposition process; and

forming a cathode on the electron transport layer,

wherein the hole injection layer is formed by forming a membrane from a solution containing a p-type oxide semiconductor mixed into a solvent.

Certain embodiments of the invention are described below in further detail. It is to be clearly understood that the embodiments described below are merely intended to explain the invention and are not to limit the scope of protection.

### Example Embodiments

As described below, a hole injection layer was formed using a p-type oxide semiconductor instead of PEDOT:PSS.

Here, it may be preferable to set the gallium content within the p-type oxide semiconductor to be 10 to 70 atomic percent.

Also, the solvent was formed by vigorously mixing ethylene glycol and acetonitrile under a general atmosphere, and a p-type oxide semiconductor was added in a concentration of 0.2M/16 to the solvent to create a mixed solution.

This solution was printed over the anode in a nitrogen environment.

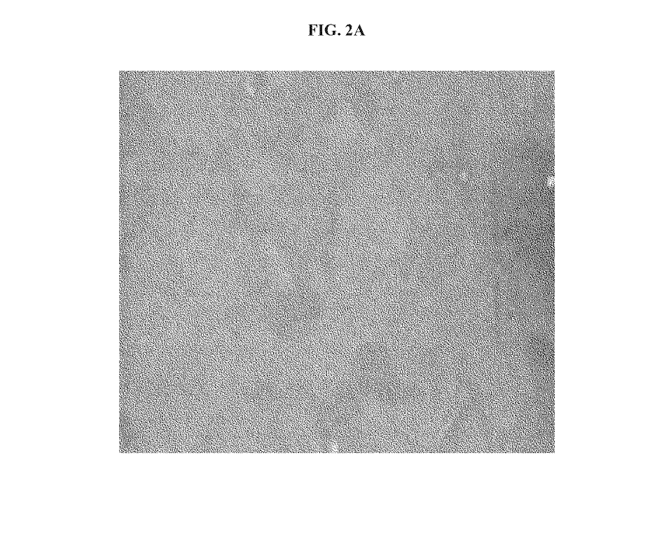

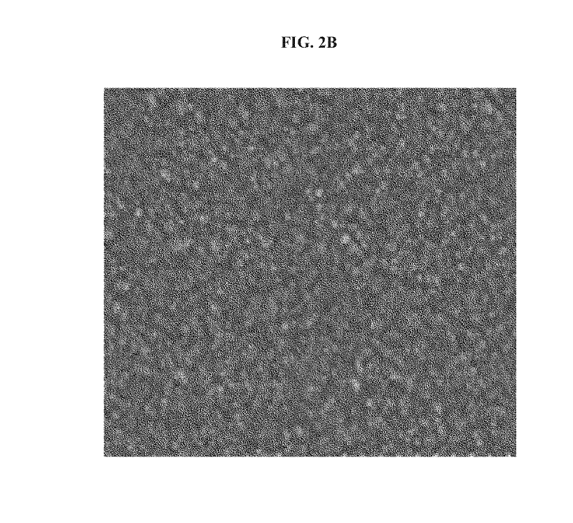

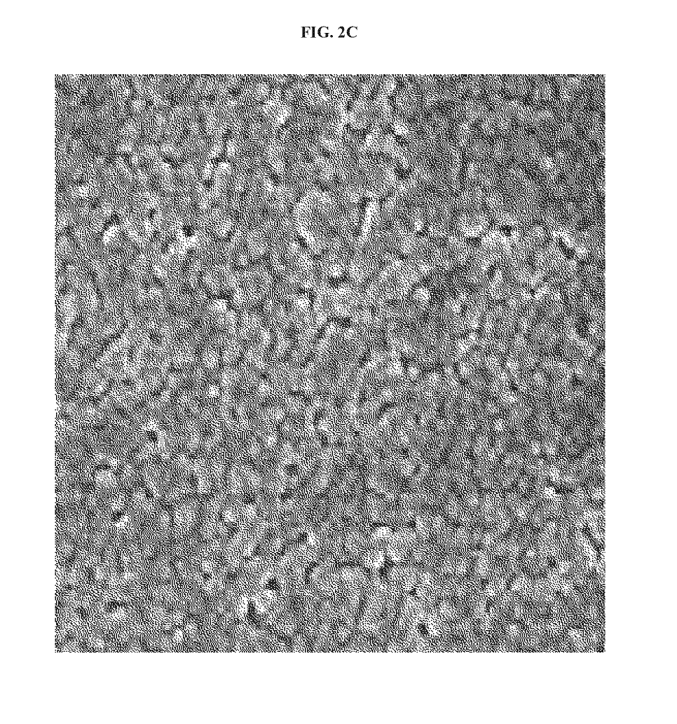

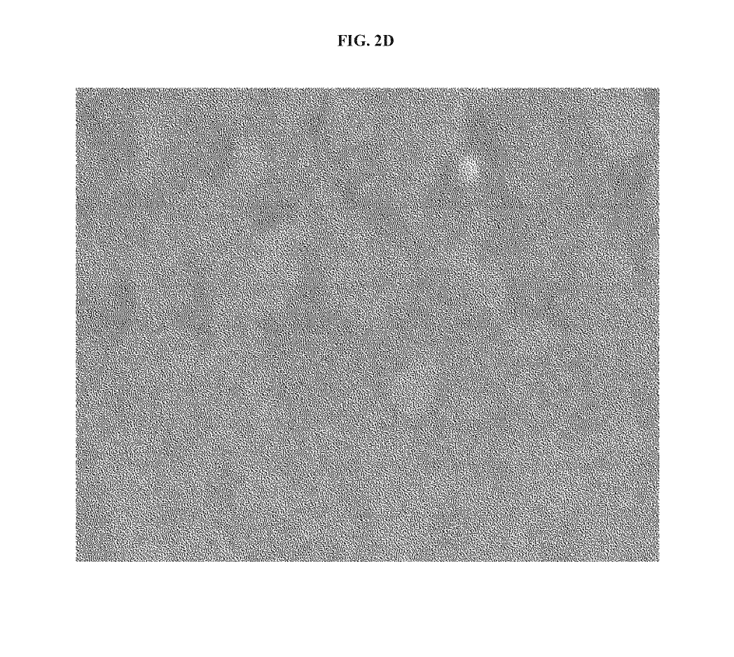

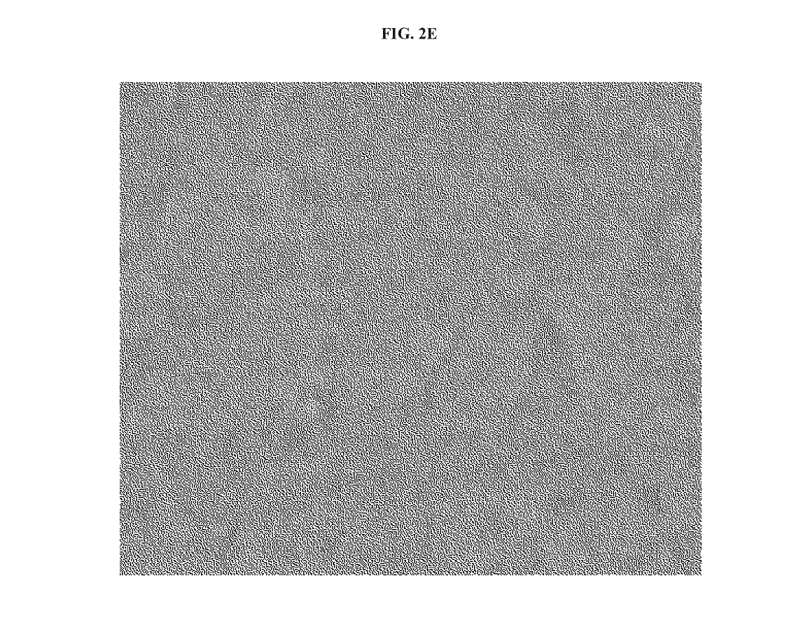

FIG. 2A to FIG. 2E illustrate the surfaces of thin films using a p-type oxide semiconductor according to an embodiment of the invention and PEDOT:PSS.

FIG. 2A to FIG. 2E show thin films using a p-type oxide semiconductor that were heat treated at 100° C., 200° C., and 300° C. and UV treated, respectively, while drawing (e) shows the PEDOT:PSS thin film.

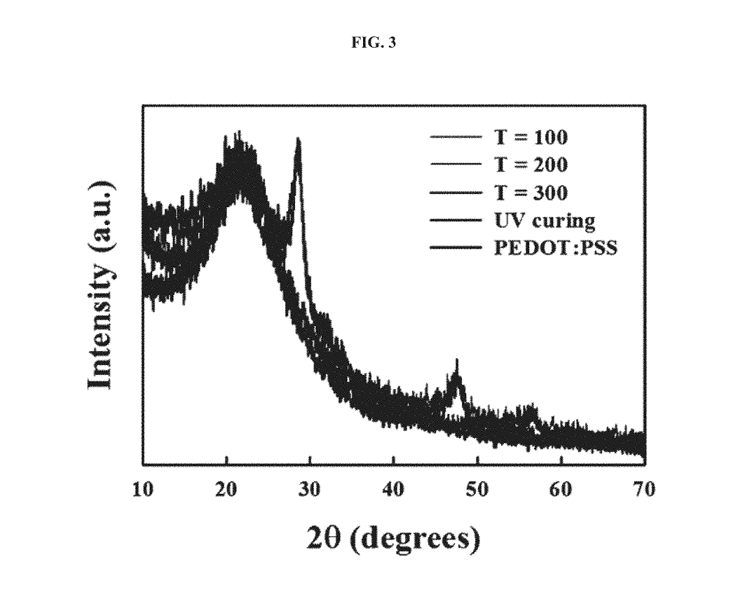

FIG. 3 shows XRD (X-ray diffraction) results for cases using a p-type oxide semiconductor according to an embodiment of the invention and a case using PEDOT:PSS.

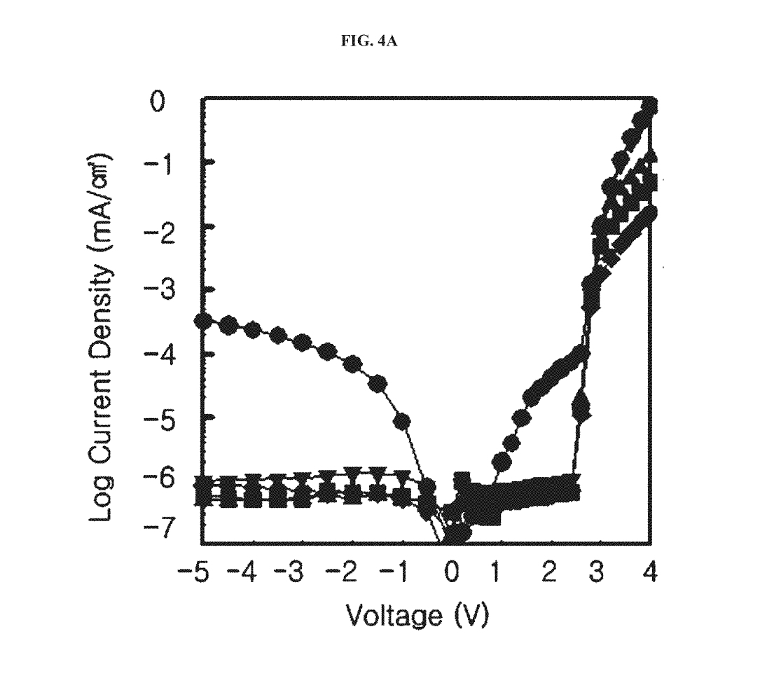

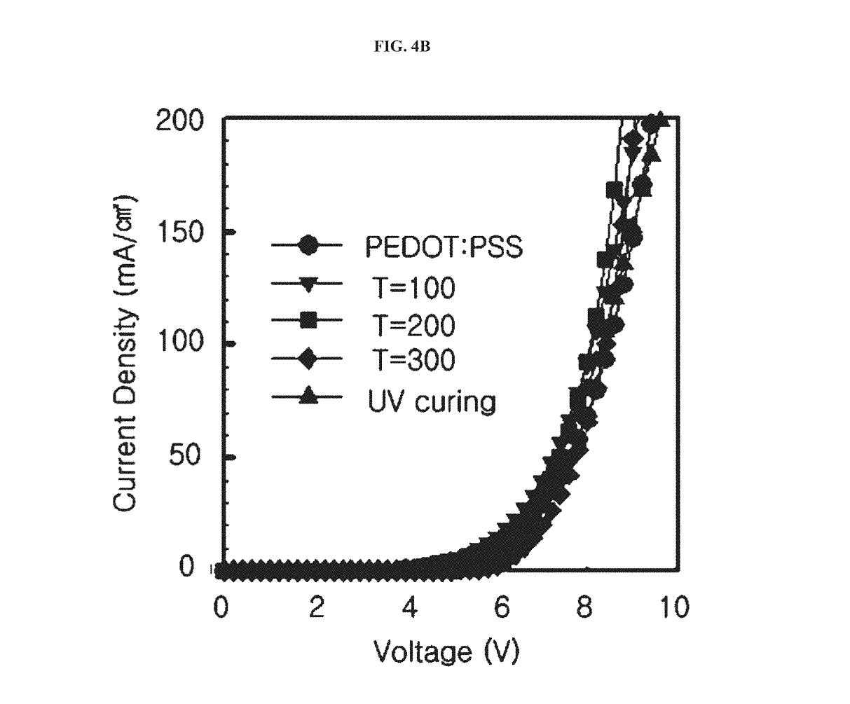

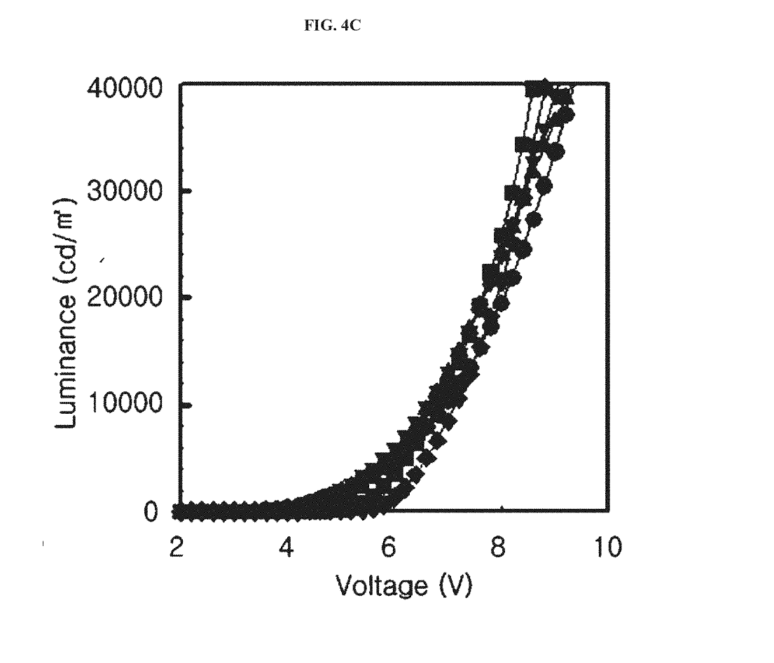

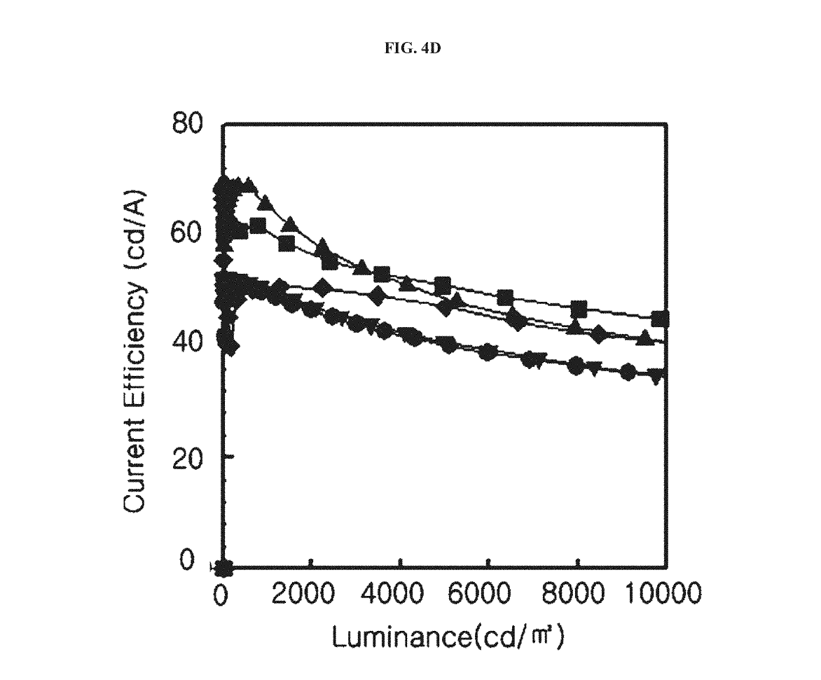

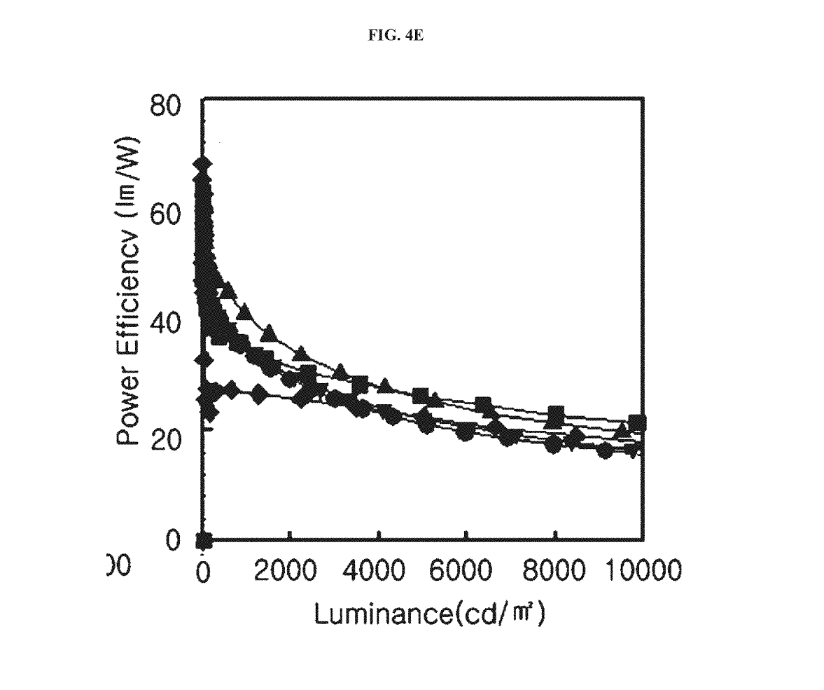

FIG. 4A to FIG. 4E and Table 1 show current-voltage-luminance properties of OLED's for cases using a p-type oxide semiconductor according to an embodiment of the invention and a case using PEDOT:PSS.

FIG. 4A and FIG. 4B show current-voltage properties, FIG. 4C shows luminance-voltage properties, FIG. 4D shows current efficiency properties, and FIG. 4E shows power efficiency properties.

TABLE 1

| | | | Maximum | | |
|---|---|---|---|---|---|
| | | | C/E | P/E | Luminance |
| | V_{T} (V) | V_{D} (V) | (cd/A) | (lm/W) | (at 8 V) |
| PEDOT:PSS | 2.82 | 4.49 | 51.3 | 50.32 | 19530 |
| CuS—GaSnO-100° C. | 2.83 | 4.5 | 52.33 | 55.32 | 24030 |
| CuS—GaSnO-200° C. | 2.85 | 5.47 | 62.18 | 63.16 | 25900 |
| CuS—GaSnO-300° C. | 2.96 | 5.91 | 69.2 | 68.2 | 21500 |
| CuS—GaSnO-UV | 2.82 | 5.02 | 68.78 | 61.46 | 24130 |
| curing | | | | | |

Referring to FIG. 4A to FIG. 4E and Table 1, when only PEDOT:PSS was used for the hole transport layer, the current efficiency was 51.30 cd/A, and the power efficiency was 50.32 lm/W.

In comparison, when a p-type oxide semiconductor was used, it can be seen that the current efficiency and the power efficiency were improved to values of up to 69.20 cd/A and 68.20 lm/W.

Also, it can be observed that current efficiency and power efficiency were increased when heat treatment was performed.

However, it can be seen that when the heat treatment temperature is increased, the driving voltage increases together whereas the luminance decreases. In consideration of this, an hole injection layer optimal for an OLED can be prepared when the heat treatment temperature is 150° C. to 250° C.; more preferably, an optimum hole injection layer can be provided when the heat treatment temperature is 200° C.

Besides the high-temperature heat treatment of the p-type oxide semiconductor, it can be observed that performance is improved also when applying UV rays at normal temperature.

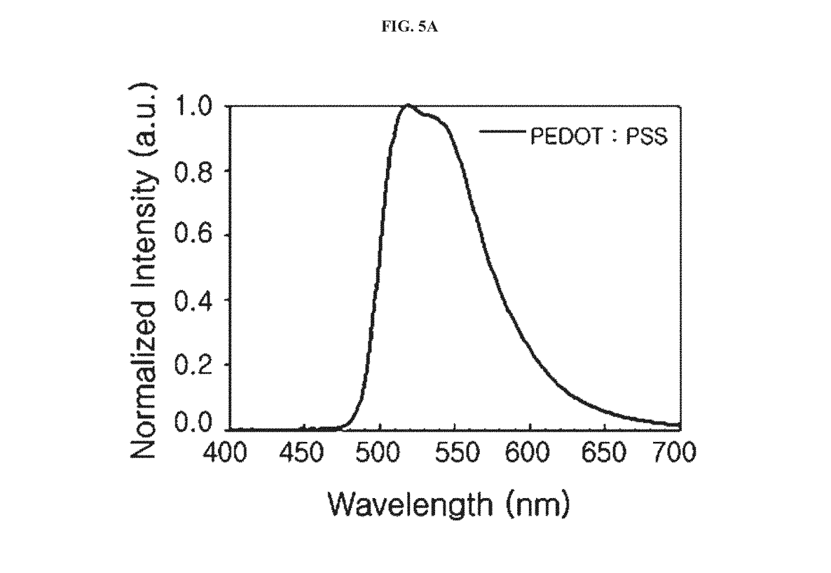

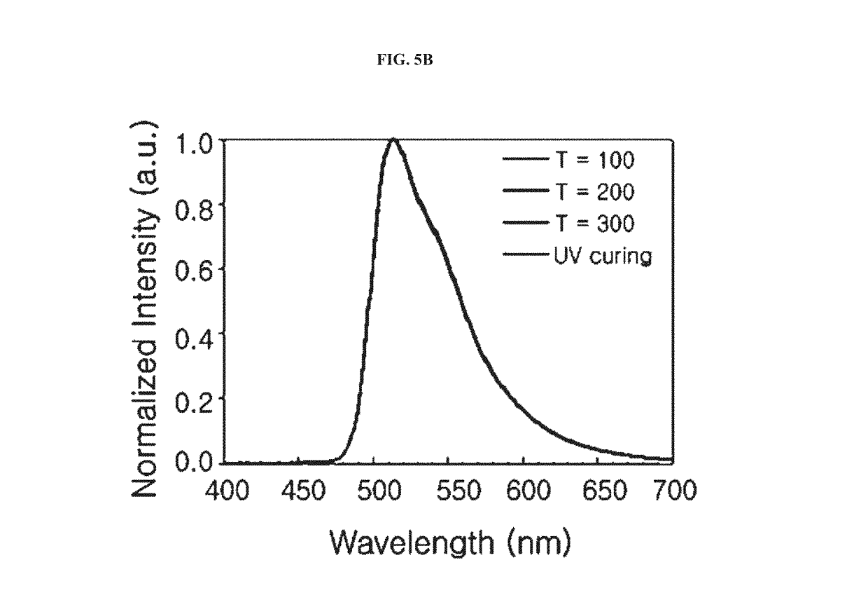

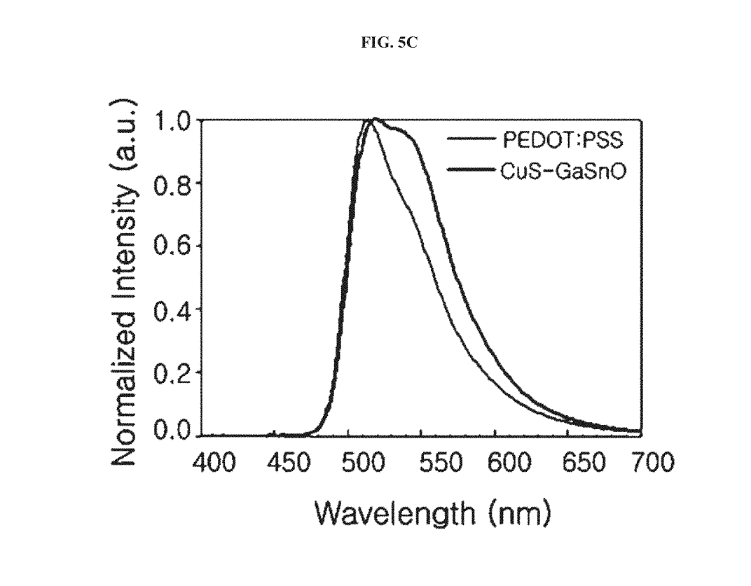

FIG. 5A to FIG. 5C show spectrum properties of OLED's for cases using a p-type oxide semiconductor according to an embodiment of the invention and a case using PEDOT:PSS.

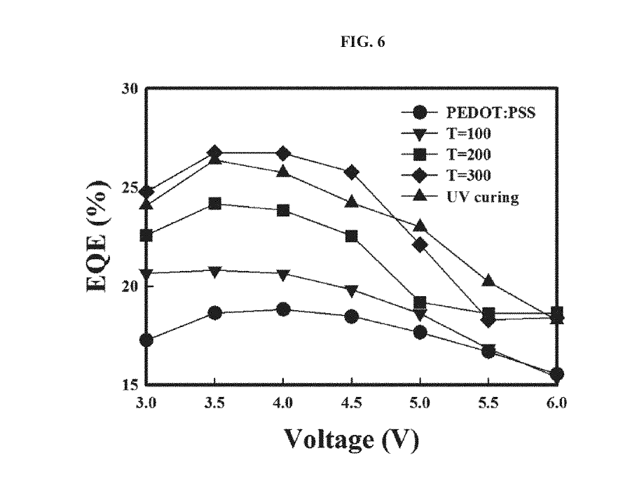

FIG. 6 and Table 2 show external quantum efficiency properties of OLED's for cases using a p-type oxide semiconductor according to an embodiment of the invention and a case using PEDOT:PSS.

TABLE 2

| | EQE (%) |
|---|---|
| PEDOT:PSS | 18.83 |
| CuS—GaSnO-100° C. | 20.79 |
| CuS—GaSnO-200° C. | 24.17 |
| CuS—GaSnO-300° C. | 26.77 |
| CuS—GaSnO-UV curing | 26.39 |

Referring to FIG. 6 and Table 2, it can be seen that the external quantum efficiency (EQE) is higher for the case where the hole injection layer is formed by printing a p-type oxide semiconductor, compared to the case where PEDOT:PSS is used.

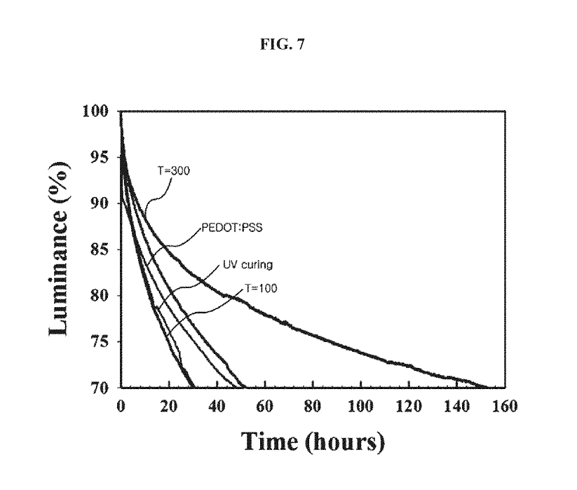

FIG. 7 and Table 3 show life expectancy properties of OLED's for cases using a p-type oxide semiconductor according to an embodiment of the invention and a case using PEDOT:PSS.

TABLE 3

| | L70 Lifetime (h) |
|---|---|
| PEDOT:PSS | 48.14 |
| CuS—GaSnO-100° C. | 30.14 |
| CuS—GaSnO-200° C. | 51.95 |
| CuS—GaSnO-300° C. | 152.77 |
| CuS—GaSnO-UV curing | 30.84 |

Referring to FIG. 7 and Table 3, it can be seen that the life expectancy of an OLED is increased in cases where the hole injection layer is formed by printing a p-type oxide semiconductor, compared to the case where PEDOT:PSS is used.

In the descriptions that follow, the procedures for preparing a p-type oxide semiconductor according to an embodiment of the invention are considered in further detail.

### Preparation of Precursor Solution

A precursor solution was prepared by dissolving CuCl₂, NH₂CSNH₂ (Thiourea), Ga(NO₃)₃.xH₂O (gallium nitrate hydrate), and SnCl₂ in an acetonitrile and ethylene glycol solvent under a nitrogen environment.

### Forming of Active Layer

The precursor solution prepared as above was applied by spin coating and heat treated for approximately 1 minute on a 240° C. hot plate or was applied by inkjet printing on a 60° C. substrate to form an active layer.

### Heat Treatment

The active layer formed by spin coating or inkjet printing as above was annealed for approximately 1 hour at a temperature of 300° C. under a nitrogen atmosphere.

### Analysis of Semiconductor Oxide

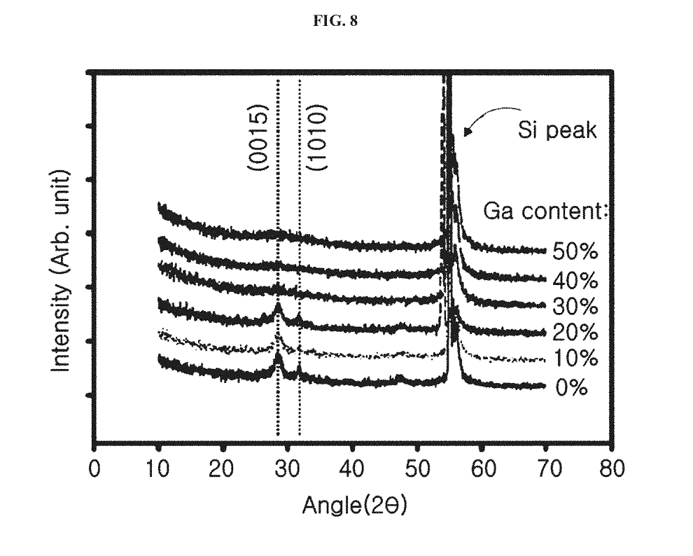

FIG. 8 shows XRD results when the concentration of Ga in a CuS—GaₓSn₁₋ₓO thin film is 0˜50%.

CuS—GaₓSn₁₋ₓO is has a polycrystalline structure)(2θ=28°,32°), but when the concentration of Ga is 30% or higher, it transforms from a crystalline state to an amorphous state.

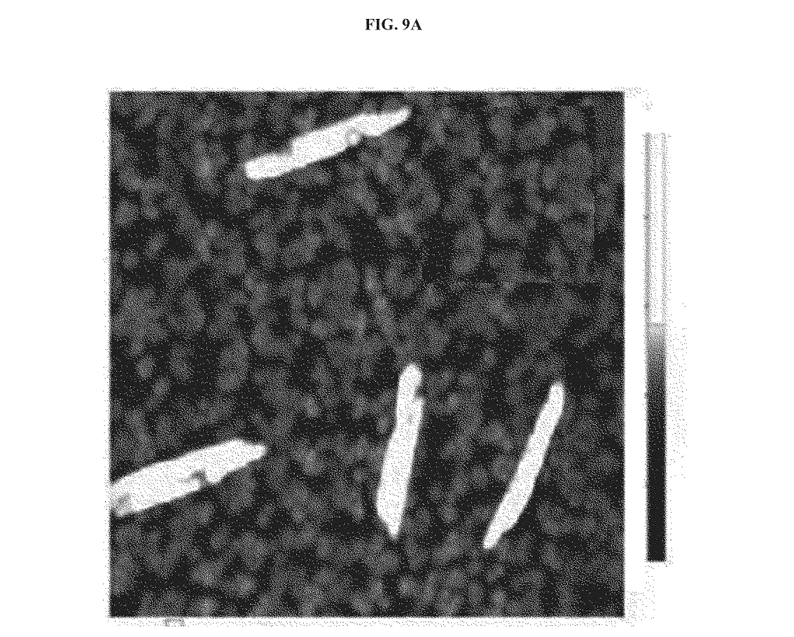

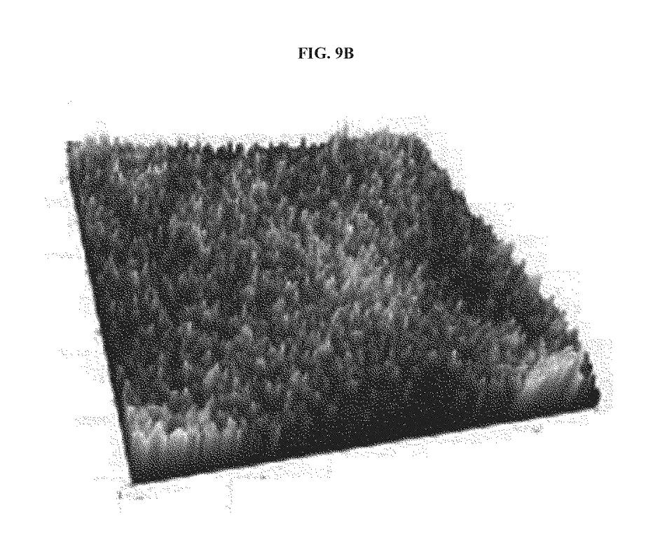

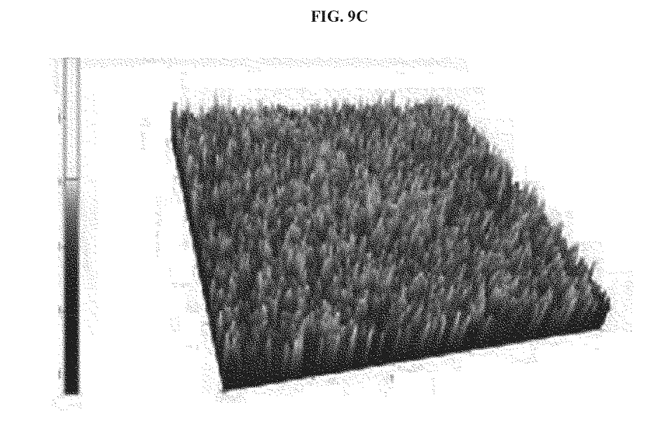

FIG. 9A to FIG. 9C show AFM images when the concentration of Ga in a CuS—GaₓSn₁₋ₓO thin film is 0%, 30%, and 50%.

When the concentration of Ga is 0%, a needle-shaped thin film was formed, with a root-mean-square (RMS) roughness value of 23˜90 nm; in this case, the thin film quality was not good.

However, as the concentration of Ga was increased, the root-mean-square roughness value decreased, resulting in improved thin film quality. At 30%, the value was 0.46˜2.5 nm, and at 50%, the value was 0.67˜3.4 nm.

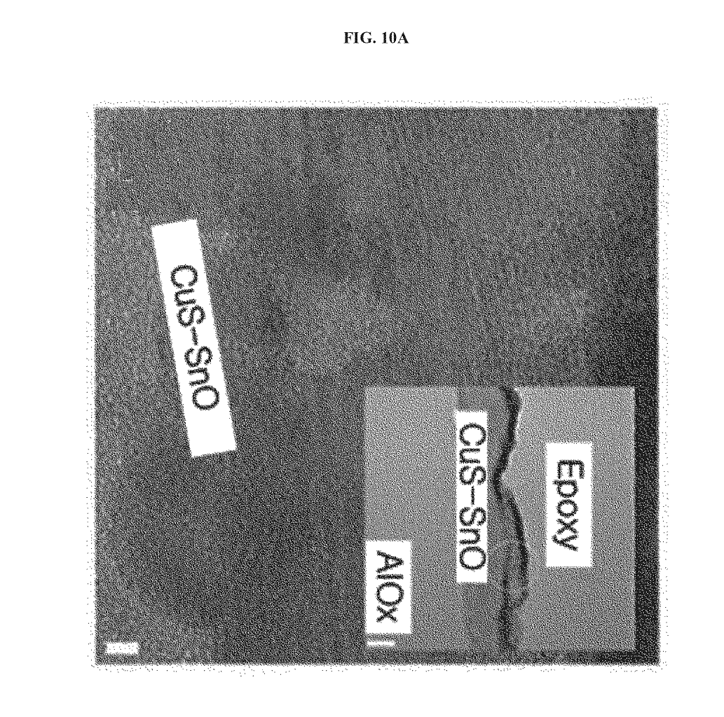

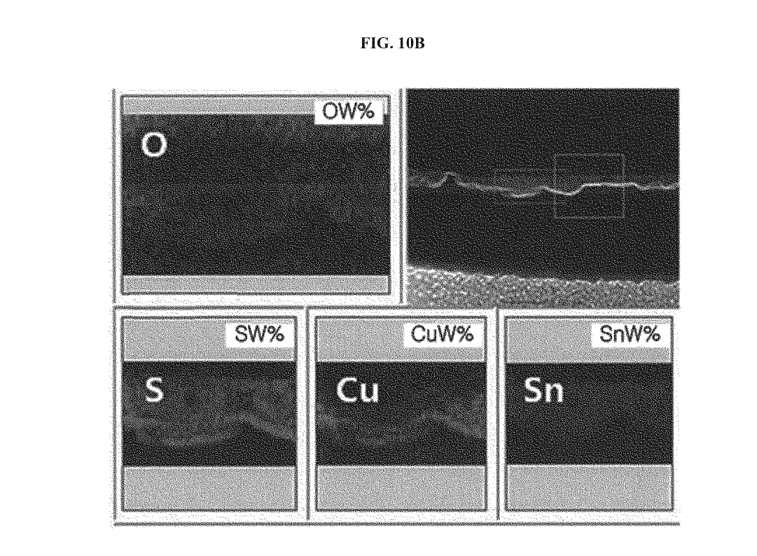

FIG. 10A and FIG. 10B show (a) a TEM image and (b) atomic mapping images obtained by EDX (energy dispersive X-ray) spectroscopy for a CuS—SnO thin film. The CuS—SnO thin film includes Cu, S, Sn, and O, and exhibits a crystalline structure of a non-uniform thin film state. This agrees with the observation from the XRD results of FIG. 8 in which a crystalline peak is present when Ga is 0%.

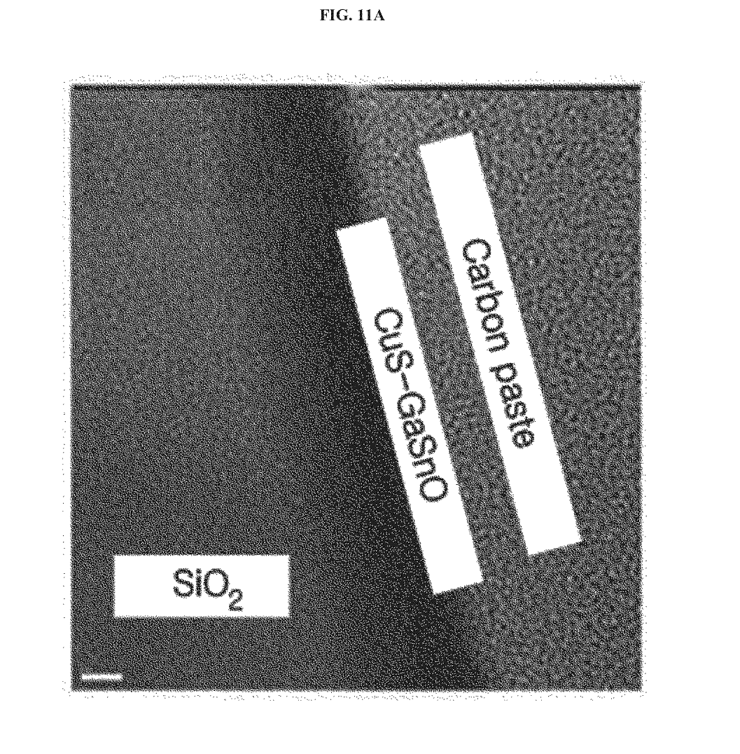

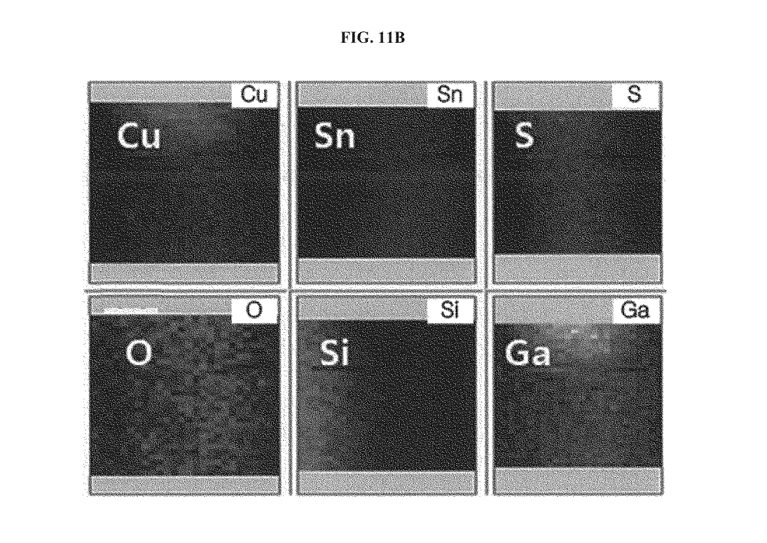

FIG. 11A and FIG. 11B show (a) a TEM image and (b) atomic mapping images obtained by EDX (energy dispersive X-ray) spectroscopy for a CuS—Ga_{0.5}Sn_{0.5}O thin film. The CuS—Ga_{0.5}Sn_{0.5}O thin film includes Cu, S, Sn, and O, and exhibits a uniform amorphous structure.

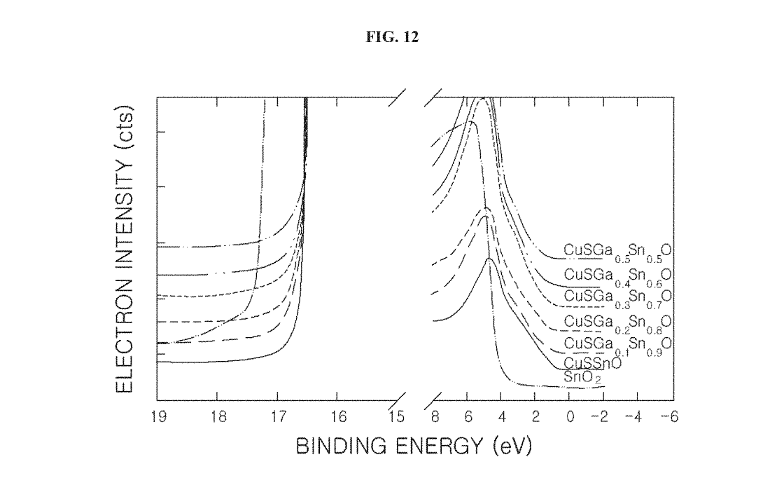

FIG. 12 and Table 4 show values of the UPS (ultraviolet photospectroscopy) and work function, the difference between the Fermi level and valence band, and the ionization potential of SnO₂ and CuS—GaₓSn₁₋ₓO.

TABLE 4

| Material | Workfunction (eV) | EF-EVBM (eV) | Ip (eV) |
|---|---|---|---|
| SnO₂ | 3.96 | 3.56 | 7.52 |
| CuS—SnO | 4.64 | 0.73 | 5.37 |
| CuS—Ga_{0.1}Sn_{0.9}O | 4.63 | 0.94 | 5.57 |
| CuS—Ga_{0.2}Sn_{0.8}O | 4.61 | 0.93 | 5.54 |
| CuS—Ga_{0.3}Sn_{0.7}O | 4.66 | 0.93 | 5.59 |
| CuS—Ga_{0.4}Sn_{0.6}O | 4.7 | 0.99 | 5.69 |
| CuS—Ga_{0.5}Sn_{0.5}O | 4.7 | 1.02 | 5.72 |

Regarding the CuS—GaₓSn₁₋ₓO thin films, crystalline thin films (Ga<0.3) exhibited work function values of 4.63 and 4.61 eV, while amorphous thin films (Ga≧0.3) exhibited high values of ≧4.66 eV.

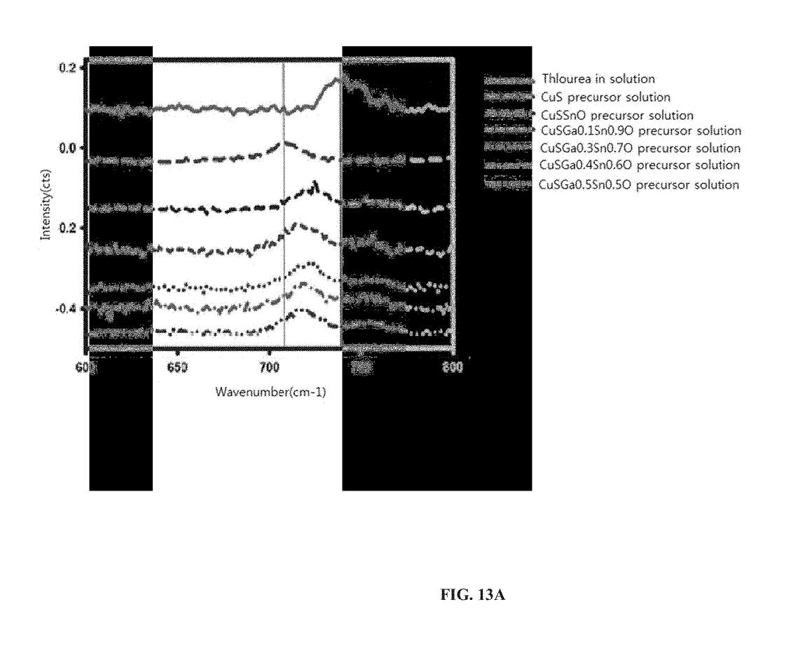

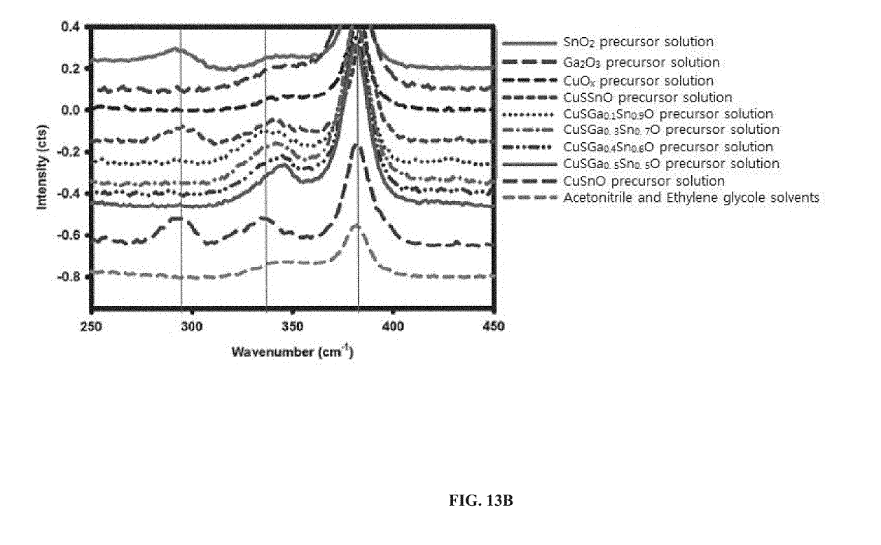

FIG. 13A and FIG. 13B show Raman spectrum results of p-type precursor solutions. FIG. 13A represents the Raman spectrum results of a p-type semiconductor solution in the region of 600-800 cm⁻¹, where a [Cu(Tu)₃]n polymer was formed at 710 cm⁻¹, and when Ga or Sn is added, the wavenumber moves to 720 cm⁻¹, and this shows that Ga or Sn is present in the solution.

FIG. 13B represents the Raman spectrum results of a p-type semiconductor solution in the region of 250-450 cm⁻¹. It can be seen that Sn is present at 290 cm⁻¹ and that Sn and Cu are present at 340 cm⁻¹. When Tu or Ca is added to the solution, the wavenumber moves to 349 cm⁻¹, and this shows that Tu is present in the solution.

The present invention set forth above is not limited to the embodiments described above or the appended drawings. It should be apparent to the skilled person that various substitutions, additions, and modifications are possible without departing from the technical spirit of the invention.

1. An organic light emitting diode comprising an anode, a hole injection layer, a hole transport layer, a light emitting layer, an electron transport layer, and a cathode,
wherein the hole injection layer is a p-type oxide semiconductor containing Ga.

2. The organic light emitting diode of claim 1, wherein the p-type oxide semiconductor includes the Ga contained in CuS and SnO.

3. The organic light emitting diode of claim 1, wherein a content of the Ga is in a range of 10 to 70% (atomic percent) of a total composition.

4. The organic light emitting diode of claim 2, wherein the p-type oxide semiconductor comprises one or more compound expressed by any one or more of Formula 1, Formula 2, and Formula 3 below:
CuS₁₋ₓGaₓ—SnO [Formula 1]
CuSGaₓSn₁₋ₓO [Formula 2]
CuSGaₓSnO [Formula 3]
where 0<x<1 in said Formula 1, said Formula 2, or said Formula 3.

5. The organic light emitting diode of claim 1, wherein the hole injection layer is heat treated at a predetermined temperature or is UV treated.

6. The organic light emitting diode of claim 5, wherein a heat treatment temperature for the hole injection layer is within a range of 150 to 250° C.

7. An organic light emitting diode comprising an anode, a hole injection/transport layer, a light emitting layer, an electron transport layer, and a cathode,
wherein the hole injection/transport layer is a p-type oxide semiconductor containing Ga.

8. A preparation method for an organic light emitting diode, the preparation method comprising:
forming an anode on a substrate by way of a vacuum deposition process;
forming a hole injection layer on the anode by way of a solution process;
forming a hole transport layer on the hole injection layer by way of a vacuum deposition process;
forming a light emitting layer on the hole transport layer by way of a vacuum deposition process;
forming an electron transport layer on the light emitting layer by way of a vacuum deposition process; and
forming a cathode on the electron transport layer,
wherein the hole injection layer is formed by forming a membrane from a solution containing a p-type oxide semiconductor mixed into a solvent.

9. The preparation method of claim 8, wherein the p-type oxide semiconductor includes the Ga contained in CuS and SnO.

10. The preparation method of claim 8, wherein a content of the Ga is in a range of 10 to 70% (atomic percent) of a total composition.

11. The preparation method of claim 10, wherein the p-type oxide semiconductor comprises one or more compound expressed by any one or more of Formula 1, Formula 2, and Formula 3 below:
CuS₁₋ₓGaₓ—SnO [Formula 1]
CuSGaₓSn₁₋ₓO [Formula 2]
CuSGaₓSnO [Formula 3]
where 0<x<1 in said Formula 1, said Formula 2, or said Formula 3.

12. The preparation method of claim 8, wherein the solvent comprises at least one of acetonitrile, DI water, alcohol, cyclohexane, toluene, and an organic solvent mixed in a proportion of 5 to 50 volume percent into ethylene glycol.

13. The preparation method of claim 8, wherein the p-type oxide semiconductor is prepared by a sequence of:
(a) preparing a precursor solution containing Cu, S, M, and Ga (where said M is one or more compound selected from a group consisting of SnO, ITO, IZTO, IGZO, and IZO);
(b) coating the precursor solution on a substrate; and
(c) heat treating the coating layer.

14. The preparation method of claim 12, wherein the precursor solution contains [CuTu₃]Cl.

15. The preparation method of claim 14, wherein the precursor solution contains Thiourea.

16. The preparation method of claim 8, wherein the hole injection layer is heat treated at a predetermined temperature or is UV treated.

17. The preparation method of claim 16, wherein a heat treatment temperature for the hole injection layer is within a range of 150 to 250° C.
